(19) United States
(12) Patent Application Publication
(10) Pub. No.: US 2017/0007442 A1
(43) Pub. Date: Jan. 12, 2017

(54) Intraoral Orthosis Device and Method for Manufacturing

(71) Applicant: Thermal Fit, LLC, Cedar City, UT (US)

(72) Inventor: Phillip Dietz, Saint George, UT (US)

(73) Assignee: Thermal Fit, LLC, Cedar City, UT (US)

(21) Appl. No.: 14/797,098

(22) Filed: Jul. 11, 2015

(51) Int. Cl. A61F 5/56

(52) U.S. Cl. CPC A61F 5/566

(57) ABSTRACT

An intraoral orthosis device that has a maxillary and mandibular bite tray connect by a telescoping arm or releasable fasteners. The configuration of the telescoping arm makes it very easy for a user to adjust the length of the telescoping arm. Another object of the invention is the use of an anchor that attaches the telescoping arm to the maxillary or mandibular bite. Yet another object of the invention is a spacer that allows the user to easily adjust the positions of the telescoping arms.

Accordingly another aspect of the present invention is to provide a method to manufacture an intraoral orthosis device. The method utilizes an analyzer, an articulator, and a sander. The new method ensures that the shape and angles of the intraoral orthosis device meet the specification while reducing the amount to time to manufacture the device.

### FIELD OF THE INVENTION

This invention relates to a device and method for manufacturing the device which prevents inference with normal breathing during sleeping, and more particularly to a device which alleviates snoring and sleep apnea.

### BACKGROUND

### PRIOR ART

Sleep apnea occurs when the muscles and tissues in the back of a person's throat relax and collapses the person's airway during sleep. As air attempts to pass through this collapsed airway, tissues vibrate and causes snoring. Sometimes this collapse prohibits airflow so severely that a person may briefly stop breathing. The person's body may automatically responds and wakes the person up, allowing airflow to resume. This pattern of air deprivation and breath resumptions is known as sleep apnea. Sleep apnea can cause multiple complications, including chronic fatigue, high blood pressure, depression, heart attack and more.

The United States of America, Food and Drug Administration (FDA) has approved two different devices to eliminate or reduce sleep apnea. This first device is a continuous positive airway pressure most commonly known as a CPAP. A CPAP devices opens a blocked airway by forcing air down a person's throat through a mask to keep the airway open and eliminating or reducing sleep apnea. There are several limitations to the CPAP device. Many people find a CPAP extremely uncomfortable and loud. Many users stop using the CPAP device.

The second device is an oral appliance device or a month piece. Traditionally, the intraoral orthosis devices comprised of a maxillary bite tray and a mandibular bite tray. The maxillary bite tray and the mandibular bite tray are connected by either a rod or a releasable fastening surface. The intraoral orthosis devices opens a blocked airway by sustaining the bottom jaw forward while ones sleep, maintaining the airway open, and allowing air to flow freely.

There are several limitations of the existing intraoral orthosis devices to control sleep apnea. The temporomandibular joint is a hinge joint that connects the lower jaw to the temporal bones of the skull. As the temporomandibular joint opens, the distance between the crowns of the back of the maxillary and mandibular teeth becomes increasing smaller with respects to the distance between the crowns of the front of the maxillary and mandibular teeth thus creating an angle.

Determining the angle of the temporomandibular joint is essential in constructing intraoral orthosis devices. Due to the complexity of getting the proper temporomandibular angle, manufactures build up acrylic on the crown of the back molar of the mandibular bite tray. When the patient bites down with the maxillary and mandibular bite trays inserted over the teeth, the force of biting down is placed 100% on the back molar where the buildup of acrylic was placed. This causes sore muscles, sore back molars and other problems. Many times the user may simply stop using the intraoral orthosis devices.

Yet another limitation is the placement of locking devices. Due to the technician not being able to get the proper temporomandibular angle, locking devices that connect the mandibular bite tray and the maxillary bite tray are connected on the anterior or interior of the teeth causing irritation and pain to the gums or tongue.

Yet another limitation is the material and hardware utilized to manufacture an intraoral orthosis devices. To connect the rod or any other locking device to the bite tray, technicians generally install anchors in the bite trays. The technician place an anchor on the anterior of the back molar of the mandibular bite tray and on the anterior of the canine of the maxillary bite tray by applying acrylic and acrylic liquid around the anchors, teeth and gums. With the maxillary and mandibular bite trays placed in a normal biting position, the mandibular and maxillary bite trays are connected together by a rod screwed into the anchors. If the length between the anchor placed on the back molar of the mandibular and the anchor placed on the canine of the maxillary is longer or shorter than the rod, the technician has to reconstruct the bite trays and replace the anchors with acrylic. Also, if the acrylic on the mandibular or maxillary bite trays protrude from the bite trays in a fashion that does not allow the arm to connect the mandibular bite tray and the maxillary bite tray, the technician has to either shave down the acrylic to allow the arm to connect to the bite trays or has to reconstruct the two trays building the anchors further away from the teeth. In addition, because the majority of bite trays are constructed of acrylic, it is very difficult to make adjustments.

Another limitation is the current methods of manufacturing the intraoral orthosis devices are extremely costly and time consuming. Currently, to manufacture an intraoral orthosis devices requires extensive hand work by a skilled technician. The technician will usually make several measurements of the bite trays and attempts to remove any excess material. If the technician removes too much material, the technician is required to add material and start the process over. The process of removing and adding material may take a skilled technician hours to create a single intraoral orthosis device. Due to the cost, many individuals whom would benefit from using an intraoral orthosis device cannot afford it.

Although the prior art did attempt to minimize the described limitations, the prior art did not resolve the limitation adequately. There remains a need for an intraoral orthosis device that is comfortable to wear. In addition, the device needs to be easily manufactured to decrease the cost.

### SUMMARY OF THE INVENTION

An intraoral orthosis device that has a maxillary and mandibular bite tray connected by two telescoping arms. The telescoping arm consists of an arm rod, an arm sleeve, and an arm screw. The configuration of the telescoping arm makes it very easy for a user or technician to adjust the length of the arm. Another object of the invention is the use of an anchor that attaches the telescoping arm to the maxillary or mandibular bite. Yet another object of the invention is a spacer that allows the user or technician to easily adjust the positions of the telescoping arms from the bite tray.

Accordingly, another aspect of the present invention is to provide a method to manufacture an intraoral orthosis device. The method utilizes thermoplastic material that is formed using an analyzer, an articulator, and a sander. The new method ensures that the shape and angles of the intraoral orthosis device meet the specification while reducing the manufacturing time. The new method may utilize the telescoping arms or a releasable fastener. The releasable fastener to be placed between the crown on the mandibular and maxillary bite trays from the back molar to the canine

### DESCRIPTION OF THE DRAWINGS

The invention may take form in certain parts and arrangement of parts, and preferred embodiment of which will be described in detail in the specification and illustrated in the accompany drawing, which for a part hereof:

FIG. 1 shows a prospective side view showing a telescopic arm connecting a maxillary bite tray to a mandibular bite tray with an adjustment pin;

FIG. 2 shows a prospective side view showing a releasable fastener connecting the maxillary bite tray to the mandibular bite tray;

FIG. 2A shows an enlarged view of a portion of the releasable fastener connecting the maxillary bite tray to the mandibular bite tray;

FIG. 3 shows a top prospective view of the invention relative to a user's teeth, with the telescopic arm connecting the maxillary bite tray to the mandibular bite tray, the dotted lines shows the location of an anchors imbedded in the maxillary bite tray and the mandibular bite tray;

FIG. 4 shows a top prospective view of the invention relative to a user's teeth, with the telescopic arm, an attachment screw and an spacer exploded from the maxillary bite tray and the mandibular bite tray;

FIG. 5 shows a side view of the anchor the dash lines illustrate the location of an interior threaded core;

FIG. 6 shows a top view of the anchor with multiple petals;

FIG. 7 shows side view of the spacers illustrating the different parts of the spacer comprising a bottom, a transition and a cylindrical pivot, in this Fig. the transition is fillet for the comfort of the user;

FIG. 8 shows side view of multiple spacers illustrating the varying heights of the spacers;

FIG. 9 shows a cross section and the relationship of an anchor, the spacer and an attachment screw;

FIG. 10 shows a top view of a bite tray relative to a user's teeth showing the typical location of the anchors imbedded into the bite tray;

FIG. 11 shows a side view of the telescopic arm with an arm screw, an arm sleeve, and an arm rod;

FIG. 12 shows an exploded view of the telescopic arm showing the arm screw, the arm sleeve, a bolt, the arm rod and an adjustment pin;

FIG. 13 shows a cross section of the telescopic arm and the relationship of the arm screw, the arm sleeve, the bolt and the arm rod;

FIG. 14 shows a side view of multiple arm rod and the varying lengths of the arm rods;

FIG. 15 is a flow diagram illustrating the steps of manufacturing of an intraoral orthosis device;

FIG. 16 shows a perspective side view of an articulator attached to a sander with a cast attached to the articulator and an analyzer;

FIG. 17 shows a side view of the cast attached to the articulator utilizing the analyzer to adjust the setting of the articulator;

FIG. 18 shows a perspective side view of the sander with the cast attached to the articulator and a bite tray placed on the cast;

FIG. 19. Shows a perspective bottom view of the bite tray and the cast;

FIG. 20 shows a perspective side view of a technician attaching the anchor to a cast with an adhesive substance;

FIG. 21 shows a side view of a technician applying material to the cast to create a platform;

FIG. 22 shows a prospective side view of a vacuum forming machine before the thermoplastic material is draped over the with the cast, the platform and the anchor;

FIG. 23 shows a prospective side view of the technician molding the thermoplastic material;

FIG. 24 shows a prospective side view of the technician molding the thermoplastic material;

FIG. 25 shows a prospective side view the bite tray and cast with the technician sanding and forming the bite tray to exposes the top of the anchor and forming the bite tray for the comfort of the user;

FIG. 26 shows a prospective side view of the technician installing a releasable fastener to the crow of the bite tray.

### DETAILED DESCRIPTION OF THE PREFERRED EMBODIMENTS

The following discussion describes embodiments of the invention and several variations of these embodiments. This discussion should not be construed, however, as limiting the invention to these particular embodiments. Practitioners skilled in the art will recognize numerous other embodiments as well. It is not necessary that the mill have all the features described below with regard to the specific embodiment of the invention shown in the figures.

In the following description of the invention, certain terminology is used for the purpose of reference only, and is not intended to be limiting. Terms such as “upper”, “lower”, “above”, and “below,” refer to directions in the drawings to which reference is made. Terms such as “inwards” and “outward” refer to directions towards and away from, respectively, the geometric center of the component described. Terms such as “side”, “top”, “bottom,” “horizontal,” and “vertical,” describe the orientation of portions of the component within a consistent but arbitrary frame of reference which is made clear by reference to the text and the associated drawings describing the component under discussion. Such terminology includes words specifically mentioned above, derivatives thereof, and words of similar import.

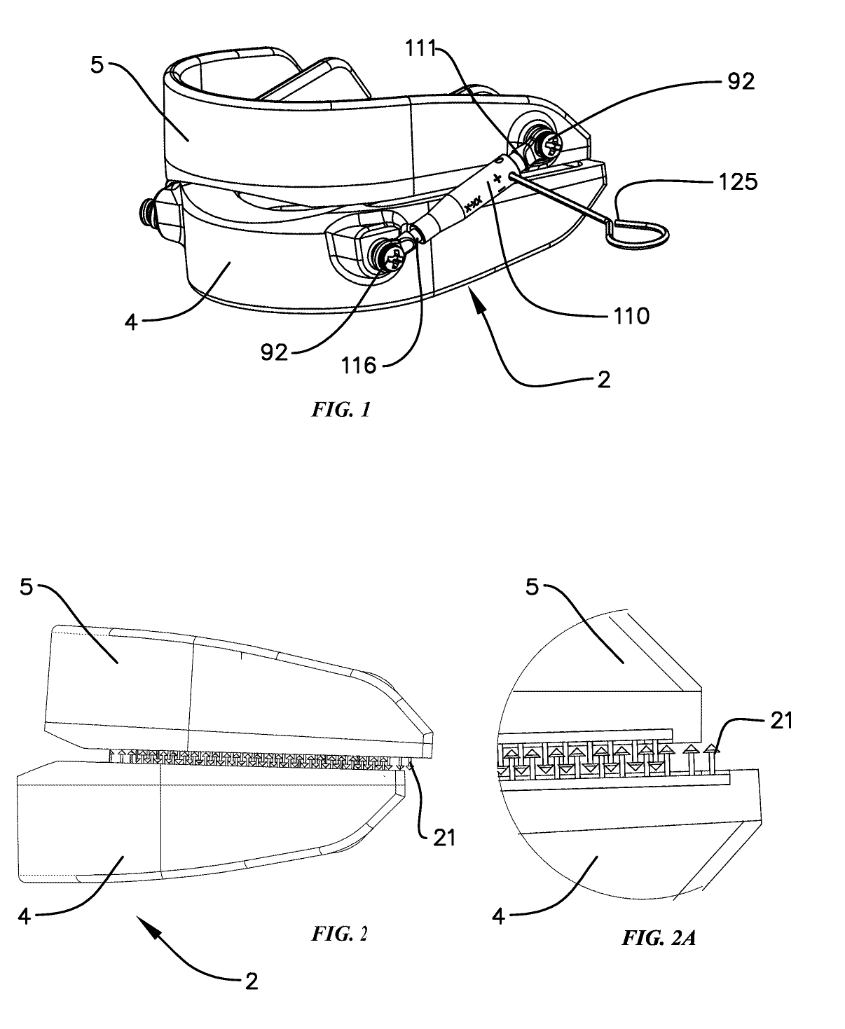

Referring generally to FIG. 1, an intraoral orthosis device 2 embodying features of the present invention comprising two bite trays 3. In practiced there will be a maxillary bite tray 5, or a mandibular bite tray 4. The maxillary bite tray 5 and the mandibular bite tray 4 are formed to substantially conform to the contours of a user's teeth. The maxillary bite tray 5 and the mandibular bite tray 4 are connected by a telescoping arm 110. As shown in FIGS. 1, 3, and 4 the telescoping arms 110 are attached to an anchor 50 embedded into the mandibular bite tray 4 and the maxillary bite tray 5 by an attachment screw 92. FIG. 2 shows another embodiment of the invention with the maxillary bite tray 5 and the mandibular bite tray 4 connected by a releasable fastener 21. The releasable fastener 21 is attached to the crown of the mandibular bite tray 4 and the maxillary bite tray 5. Generally, the releasable fastener 21 is attached by an adhesive material such as glue.

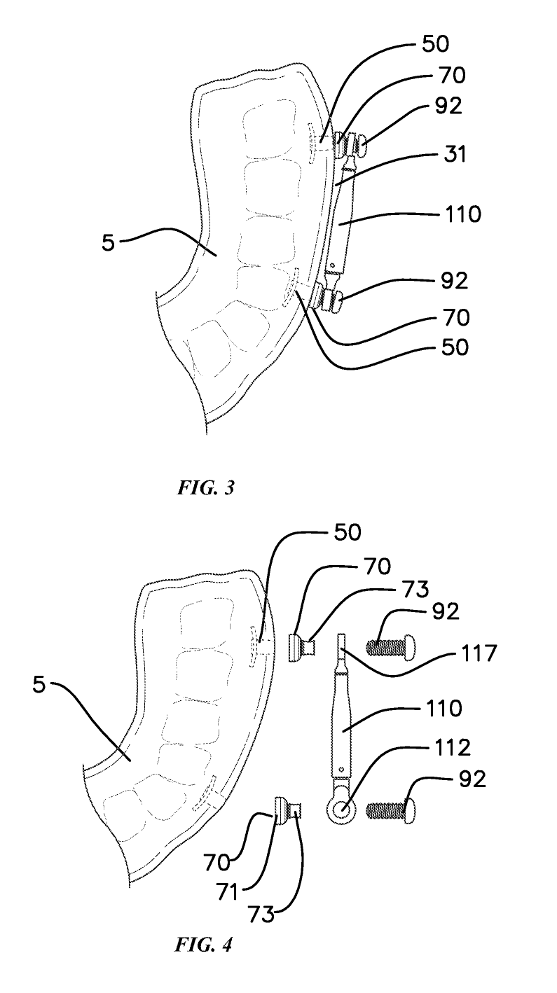

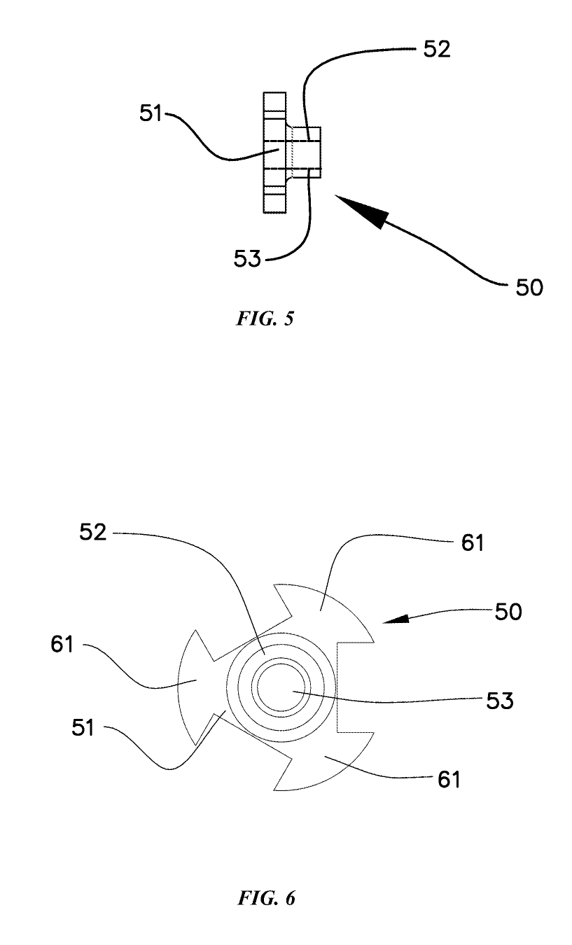

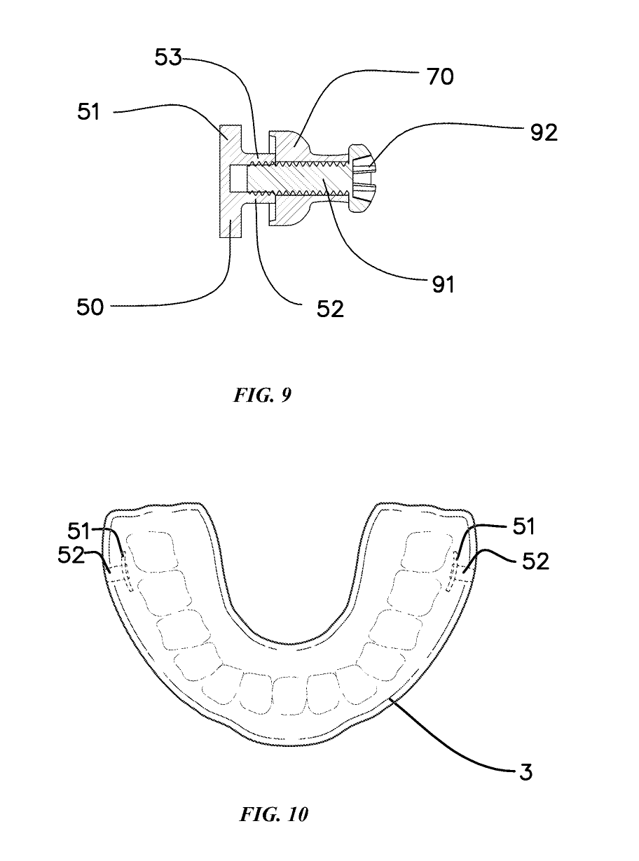

As shown in FIGS. 5 and 6, the anchor 50 comprises a base 51, a shaft 52, and an interior threaded core 53. As shown in FIGS. 3 and 4, the base 51 is imbedded into the interior of the mandibular bite tray 4 and the maxillary bite tray 5. The shaft 52 extends from the interior of the bite tray 3 to the exterior. The end of the shaft 52 is generally even with the outer surface of bite tray 3. The interior threaded core 53 is for receiving the attachment screw 92. As shown in FIG. 9, the interior threaded core 53 extends through the shaft 52 and may extend through the base 51. Generally, the anchor 50 is placed on the back molar of the mandibular bite tray 4 and near the canine of the maxillary bite tray 5. Preferably, the anchor 50 is manufactured from stainless steel, although any ridged material may be utilized. While the figures show the anchors 50 utilizing the telescoping arm 110, the anchors 50 made be utilized by any device connecting the mandibular bite tray 4 and the maxillary bite tray 5.

As shown in FIG. 6, the base 51 consists of several petals 61 extending from the longitude axis of the base 51. When the attachment screw 92 is installed, a rotational force or torque is applied to the anchor 50. The petals 61 provide additional surface area for securing the anchor 50 to the bite tray 3 to resist the rotational force. In practice, three petals 61 are utilized, however, one skilled in the art will recognize that the size, length and number of petals may vary.

As stated above, the maxillary bite tray 5 and the mandibular bite tray 4 are connected by at least one telescoping arm 110, in practice two telescoping arms 110 would be utilized. As illustrated in FIG. 12, the telescoping arm 110 includes an arm screw 111, an arm sleeve 113, a bolt 122, and an arm rod 116.

The arm screw 111 is generally a cylindrical shape rod with screw threads located along the outer diameter of the longitudinal axis. Located at the end of the arm screw 111 is an arm screw eyelet 112. The arm rod 116 is generally a cylindrical tube. The outer surface of the arm rod 116 is smooth, the inner portion has an internal threads 124 along the longitudinal axis. Located at the end of the arm rod 116 is an arm rod eyelet 117.

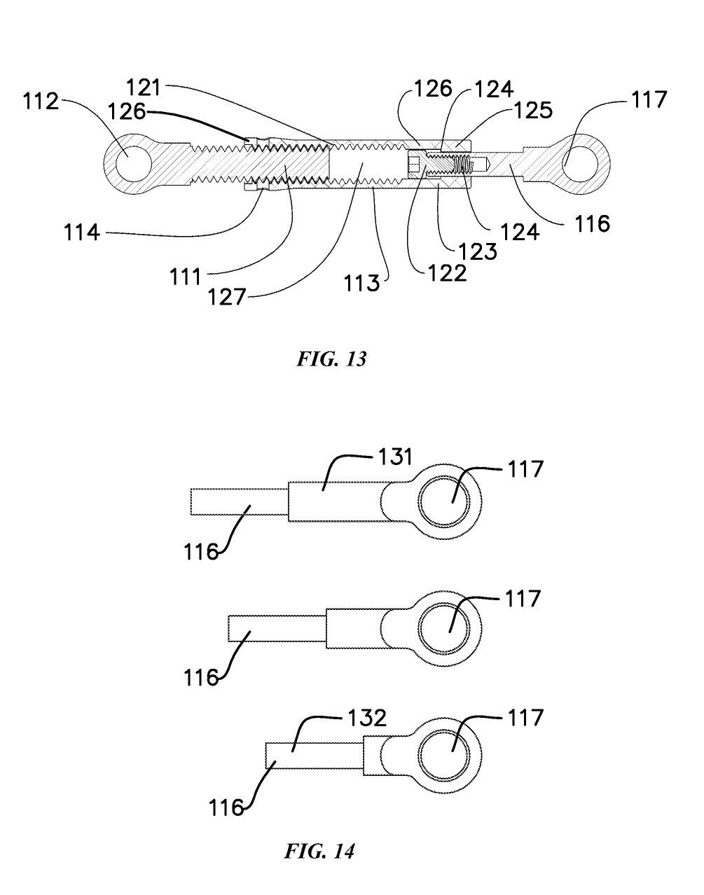

The arm sleeve 113 is generally a cylindrical shape tube. As seen in FIG. 13, the arm sleeve 113 has an interiorly threaded cylindrical portion 121 and an interiorly smooth cylindrical portion 123. The interiorly threaded cylindrical portion 121 is for receiving the arm screw 111. The interiorly smooth cylindrical portion 123 is for receiving the arm rod 116. The interiorly smooth cylindrical portion 123 is comprises of two different diameters widths creating a narrow portion 125 and a wide portion 126. The narrow portion 125 is located near the end of the arm sleeve 113. The wide portion 126 is located near the center of the arm sleeve 113. The junction at the narrow portion 125 and the wide portion 126 creates a ledge 124.

As illustrated in FIG. 13. the bolt 122 secures the arm rod 116 to the arm sleeve 113. The head of the bolt 122 has a diameter which is smaller than the wide portion 126 and a diameter larger than the narrow portion 125. The bolt 122 interactions with the ledge 124 and secures the arm rod 116 to the arm sleeve 113. An opening 127 is created between the bolt 122 and the arm screw 111. The bolt 122 and the arm rod 116 freely slide within the length of the wide portion 126. The arm rod 116 may also slide freely along the longitudinal axis of the arm sleeve 113 within the opening 127.

As seen in FIG. 12, the outer diameter of the arm sleeve 113 is uniform except at the arm sleeve expansion 126 where the outer diameter increases and flairs outwards from the longitudinal axis of the arm sleeve 113. The arm sleeve expansion 126 is located near the interiorly threaded cylindrical portion 121, as seen in FIG. 13.

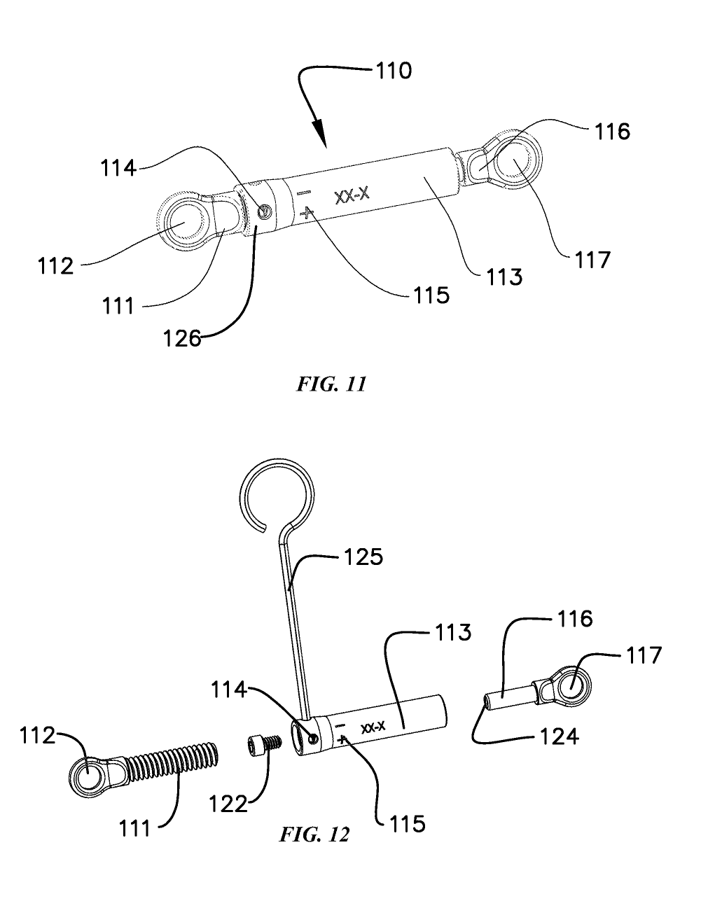

As seen in FIG. 1, the maxillary bite tray 5 and mandibular bite tray 4 are situated in the natural biting position. Because each user's mouth is unique and the location of the anchors 50 may vary, the length of the telescoping arm 110 may be adjusted. As shown in FIG. 11, the arm rod 116 and arm screw 111 maybe completely covered by the arm sleeve 113. However, as shown in FIG. 13 the arm rod 116 and arm screw 111 may extend out of the arm sleeve 113. As seen in FIG. 12, the arm sleeve expansion 123 has at least one adjustment port 114. The adjustment port 114 allows the user to place an adjustment pin 125 in the adjustment port 114. As shown in FIG. 1, when a force is applied to the adjustment pin 125, the forces causes the arm sleeve 113 to rotate. Dependent on which way the arm sleeve 113 is rotated, the arm screw 111 will either extend out of the arm sleeve 113 or descend into the arm sleeve 113. The adjustment of the depth of the arm screw 111, affects the overall length of the telescoping arm 110. Located on the outer diameter of the arm sleeve 113 is an inscription 115. The inscription 115 provides directions to the user on which way to rotate the arm sleeve 113 with the adjustment pin 125 to adjust the length.

A user or technical may also adjust the length of the telescoping arm 110, by utilizing different length arm rods 116. The length of the arm rods 116 may vary, as seen in FIG. 14, there is an extended arm rod 131 and a reduced arm rod 132. The user may extent the length of the telescoping arm 110, by utilizing the extended arm rod 131. Reduce the length of the telescoping arm 110, the user would utilize the reduce arm rod 132. By utilizing the different arms rod 116 and by adjusting the depth of the arm screw 111, the user can fine tune the length of the telescoping arm 110 for maximum comfort.

To prevent the telescoping arm 110 from contacting the bite tray 3, a clearance 31 is required. As shown in FIGS. 3 and 4, located between the telescoping arm 110 and the anchor 50 is a spacer 70. The spacer 70 allows the user or technician to create the correct clearance 31. The spacers 70 height may vary, as illustrated in FIG. 8, there is an elongated spacer 75 and a petite spacer 76. The user or technician may attach the tall spacer 75 and the petite spacer 76 to adjust the clearance 31 to the bite tray 3 as shown in FIG. 3.

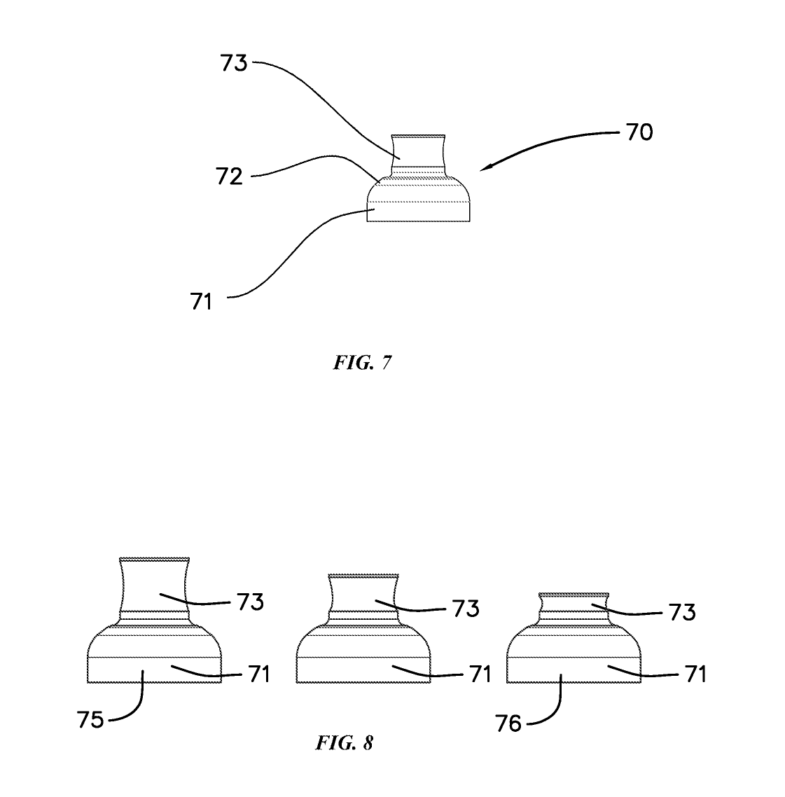

As shown in FIG. 7, the spacer 70 comprises of a bottom 71, a transition 72 and a cylindrical pivot 73. The transition 72 may be fillet for the comfort of the user. The height of the bottom 71 may vary between 0.5 mm to 20 mm. As shown in FIG. 3, the bottom 71 is located next to the bite tray 3 and the anchor 50.

The cylindrical pivot 73 is utilized to allow the telescoping arm 110 to rotate or pivot freely around the spacer 70. As shown in FIG. 3, the inner diameter of the arm screw eyelet 112 and arm rod eyelet 117 is larger than the outer diameter of the cylindrical pivot 73, such that when the cylindrical pivot 73 is placed inside the arm screw eyelet 112 or arm rod eyelet 117, the telescoping arm 110 is free to rotate and pivot. However, the space between the arm screw eyelet 112 and the cylindrical pivot 73 is small enough that it prevents the user's tissue from becoming pinched.

As shown in FIG. 9, the center of the spacer 70 has a conduit 91 for the attachment screw 92. Preferably, the spacer 70 is manufactured from stainless steel, although any ridged material may be utilized.

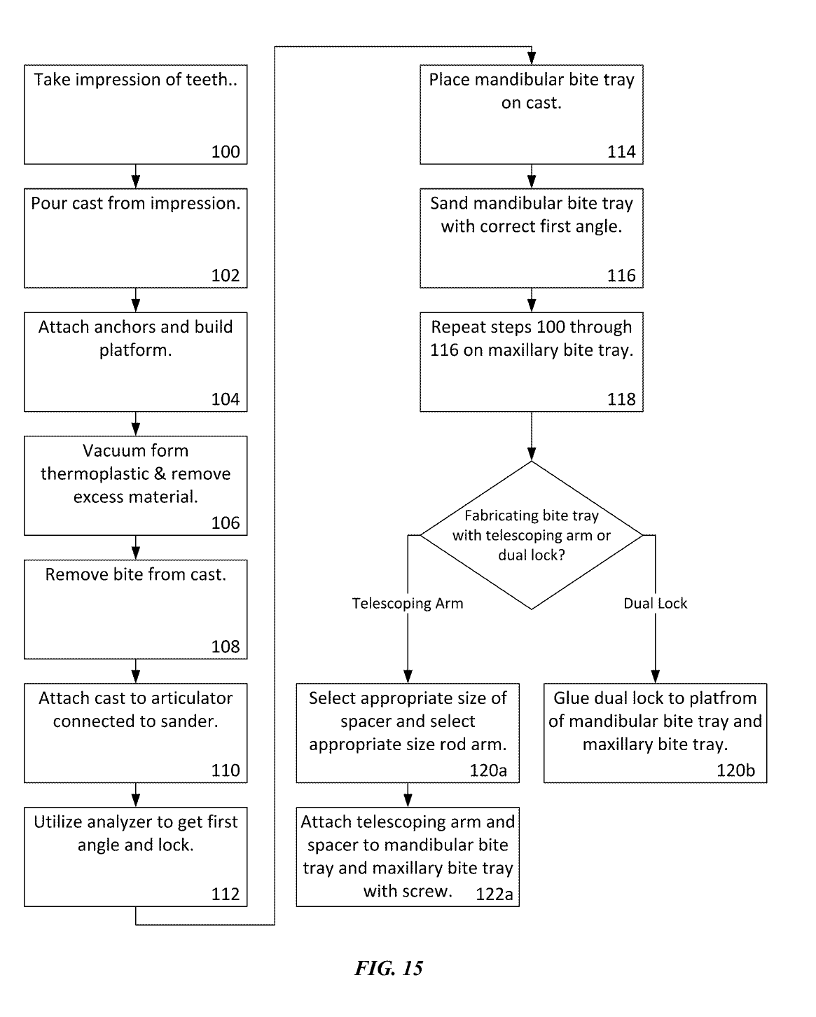

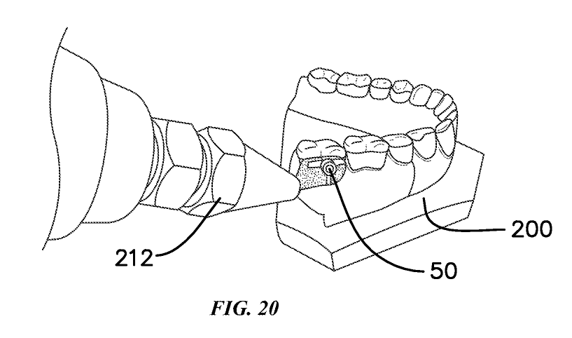

FIG. 15 illustrates the steps taken by a user or technician to manufacture the intraoral orthosis device 2. FIG. 19 shows an impression 190 is made of the user's upper and lower teeth (step 100). FIG. 20 show that from the impressions 190, a cast 200 of the mandibular teeth and the maxillary teeth are fabricated (step 102). In addition, FIG. 19, shows the technical attaching the anchors 50 to the anterior of the back molar of the mandibular cast 200. For the maxillary cast 200, the anchor 50 would be attached to the anterior of the canine The material utilized to attached the anchors 50 is generally an ethylene-vinyl acetate polymer. Ethylene-vinyl acetate is a polymer that is an elastomeric material which is soft and flexible, yet can be processed like other thermoplastics. A hot glue gun 212 is used to melt and apply the ethylene-vinyl acetate. However, other material that has the same properties as ethylene-vinyl acetate may be utilized.

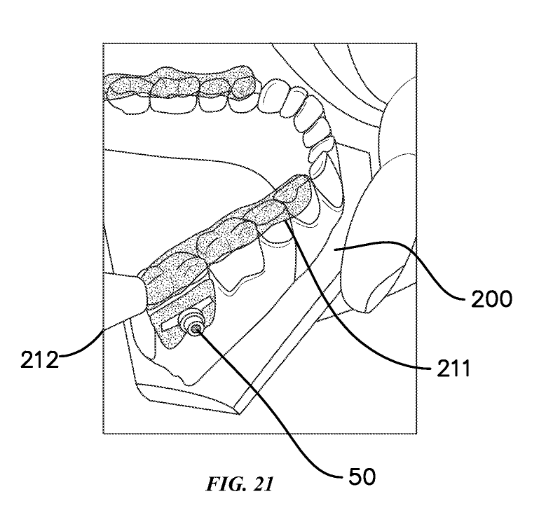

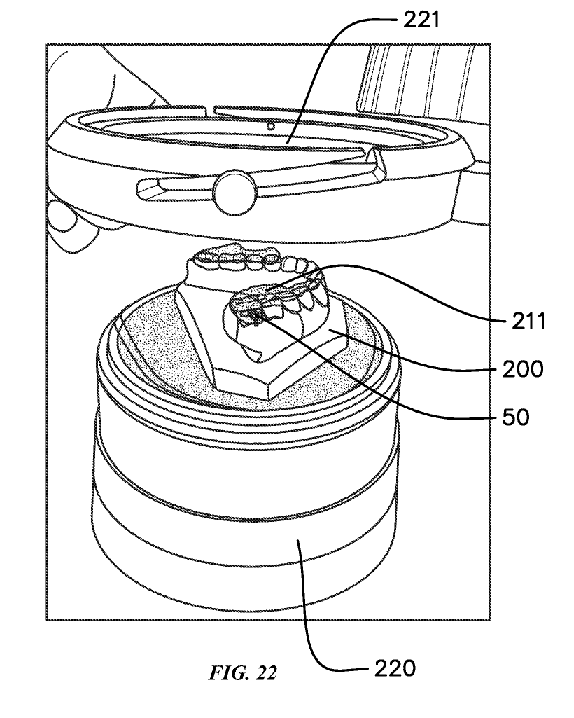

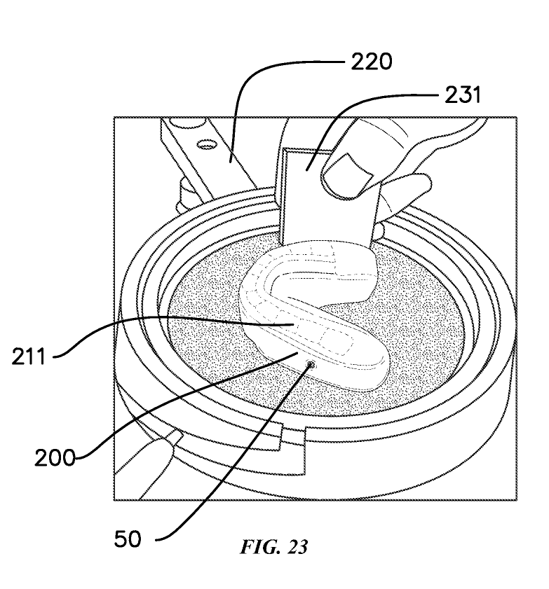

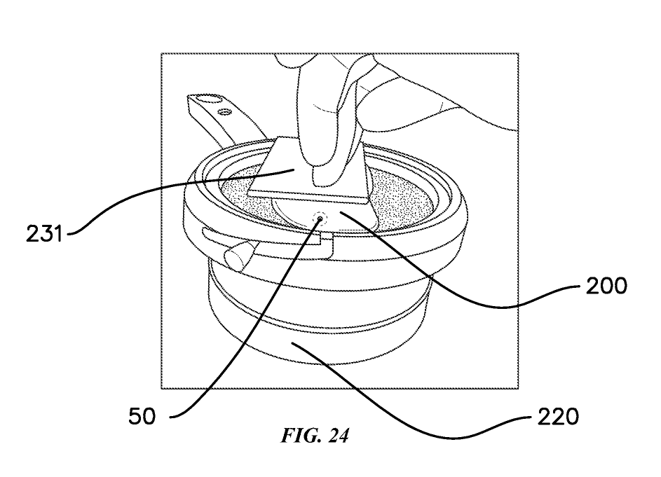

As shown in FIG. 21, the technician creates a platform 211 using ethylene-vinyl acetate on the crown of the canine back molar with the glue gun 212 (step 104). As illustrated in FIG. 22, the cast 200 with the anchors 50 and platform 211 is placed on a vacuum forming machine 220 (step 106). In a vacuum forming machine 220, heat is applied a thermoplastic material 221. As shown in FIG. 23 after the thermoplastic material 221 is heated, the thermoplastic material 221 is draped over the cast 200, anchor 50 and the platform 211. A vacuum and heat is applied to the bottom side of the thermoplastic material 221. The vacuum causes the thermal plastic material 221 to wrap and shrink tightly over the cast 200, anchor 50 and platform 211, forming the bite tray 3. The glue utilized to attach the anchor 50 and build the platform 211 fuses with the thermoplastic material 221. The anchor 50 is now embedded into the bite tray 3.

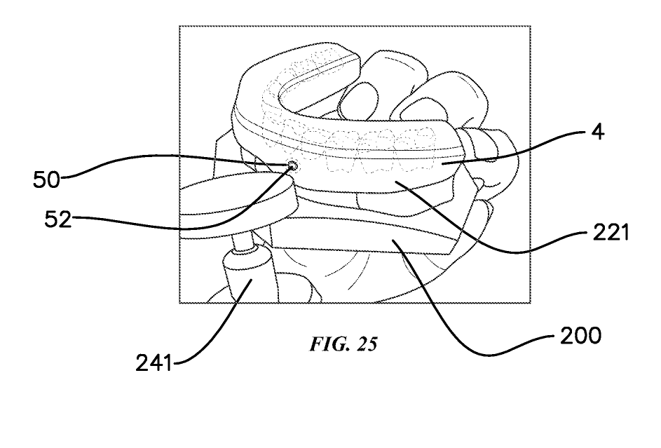

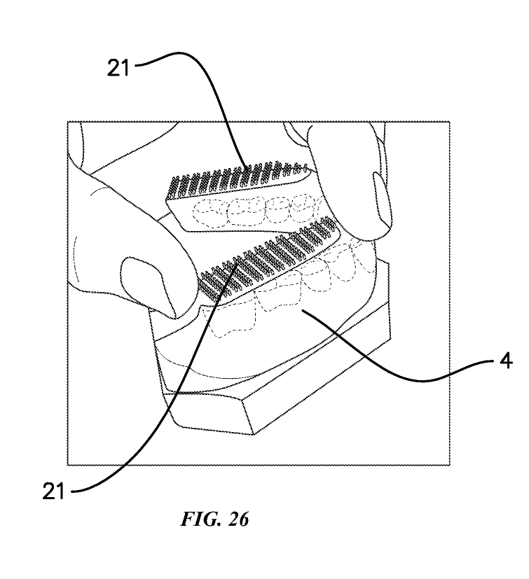

While the thermoplastic material 221 is still malleable, a technician utilizes a forming tool 231 shapes the thermoplastic to the correct shape around the cast 200, anchors 50 and platform 211 as illustrated in FIGS. 23 and 24. The cast 200 and newly formed bite tray 3 with the anchor 50 is removed from the vacuum forming machine 220.

As shown in FIG. 25, excess thermoplastic is removed and the thermoplastic material 221 covering the end of shaft 52 of the anchor 50 is removed with a dermal 241 exposing the tip of the shaft 52. The bite tray 3 is removed from the cast 200 (step 108).

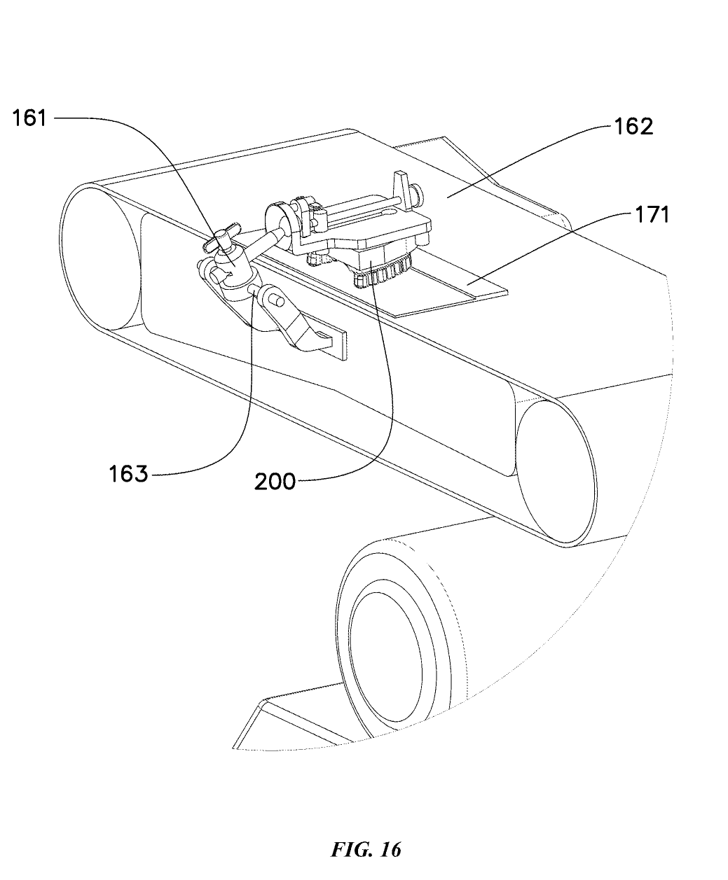

As shown in FIG. 16, the cast 200 is attached to an articulator 161 (step 110). The articulator 161 is a mechanical device used in dentistry that holds the cast 200. The articulator 161 is attached to a sander 162 by means of a hinge joint 163 that allows the technician to rotate the articulator 161 away from the sander 162.

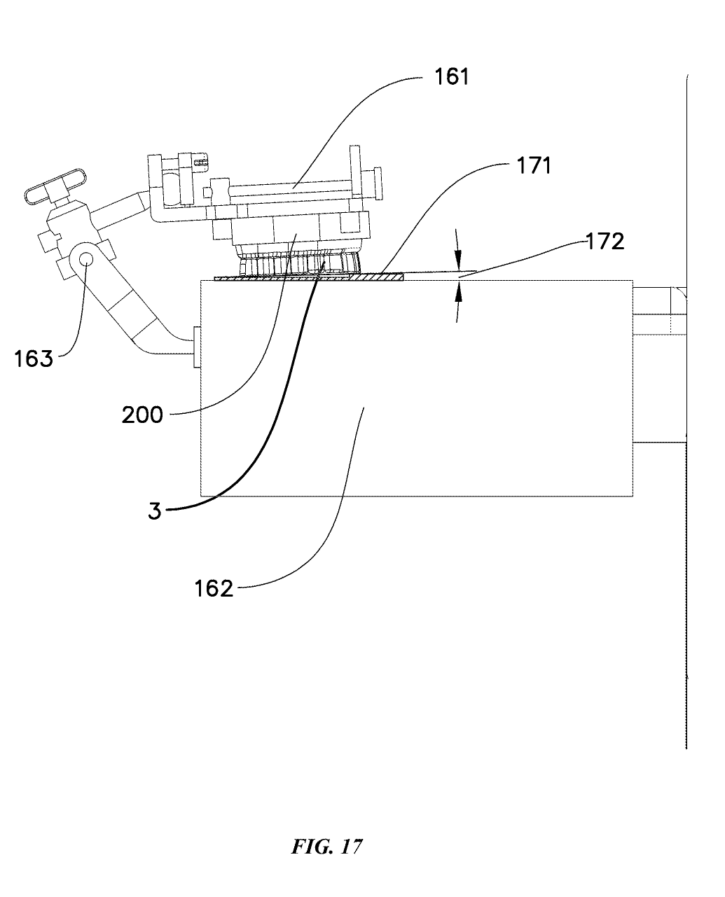

As shown in FIG. 17, utilizing the articulator 161, the crown of the mandibular cast 200 is placed against the sander 162. An analyzer 171 is placed between the sander 162 and the cast 200. The analyzer 171 creates a first angle 172 between the horizontal plain of the sander 162 and the crown of the cast 200. The first angle 172 has a similar vertex as the temporomandibular joint. Achieving the proper angle and vertex is essential for comfort to the user.

If the first angle 172 is set correctly, the maxillary bite tray 5 (FIG. 1) and the mandibular bite tray 4 has a constant contact from the back molar to the canine teeth. The preferred angle of the first angle 172 is between 0.5 and 10 degrees from the horizontal plane of the sander 162 as shown in FIG. 17. The analyzer 171 is manufactured from any ridged or semi-ridged material such as plastic or metal. The user locks the first angle 172 with articulator 116 (step 112).

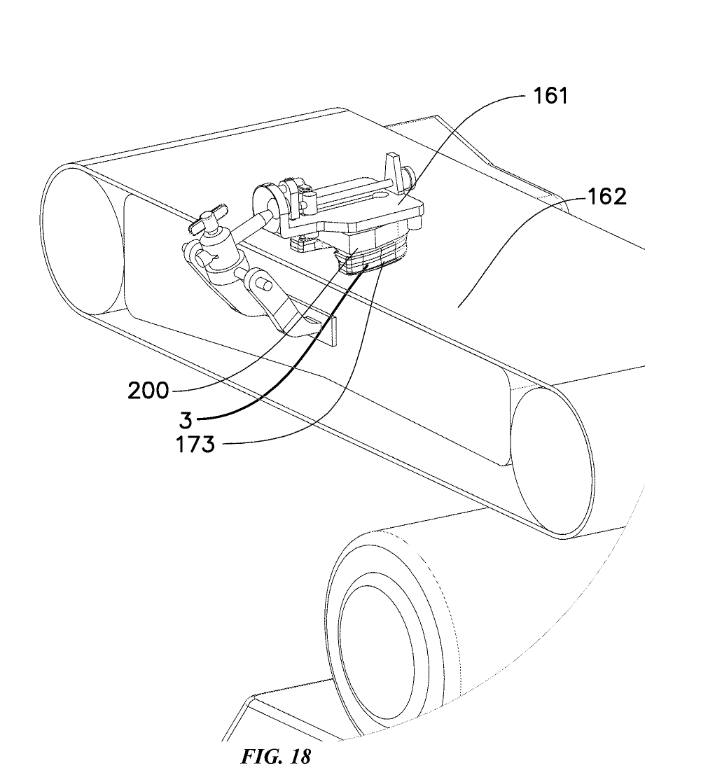

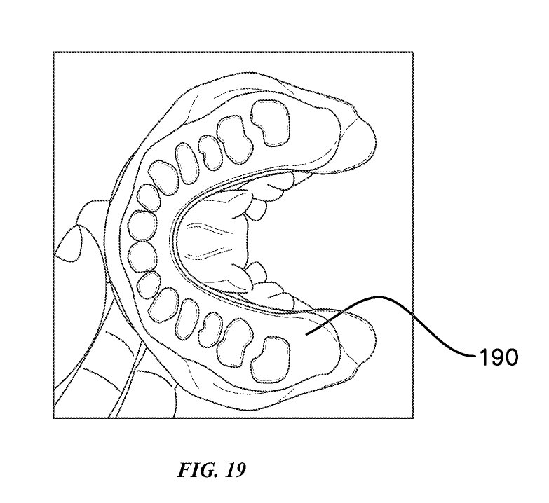

As illustrated in FIG. 18, the bite tray 3 is placed on the cast 200 and the analyzer 171 is removed (step 114). The mandibular bite tray 4 is sanded until approximately 0.5 to 2 mm of material is left between the back molar and the top of the platform (step 116). The locked articulator 161 prevents over sanding of the platform 173 and ensures the correct angle and vertex. The bite tray 3 is removed from the cast 200.

The user or technician is to repeat steps 102 through 116 to create both the maxillary bite tray 5 and the maxillary bite tray 5 (step 118).

To fabricate an intraoral orthosis device 2 with telescoping arms 110 as shown in FIG. 1, the mandibular bite tray 4 and the maxillary bite tray 5 are situation in the natural bite position. As illustrated in FIG. 3, the telescoping arms 110 and spacers 70 are connected to the mandibular bite tray 4 by inserting the attachment screw 92 through the arm rod eyelet 117 of the telescoping arm 110, through the conduit 91 of the spacer 70 and tightening the attachment screw 92 to the anchor 50. Then the telescoping arms 110 and spacers 70 are connected to the maxillary bite tray 5 by inserting the attachment screw 92 through the arm screw eyelet 112 of the telescoping arm 110, through the conduit 91 (FIG. 9) of the spacer 70 and tightening the attachment screw 92 to the anchor 50.

If the telescoping arm 110 is too short or too long to connect the mandibular bite tray 4 and maxillary bite tray 5, the length may be adjusted by employing a different length arm rod 116 or adjusting the arm sleeve 113 (step 120a).

As described above and shown in FIG. 3, to attain the clearance 31 required between the telescoping arm 110, the mandibular bite tray 4, and maxillary bite tray 5, the spacer 70 is placed between the telescoping arm 110 and the anchor 50. To adjust the width of the clearance 31, different sized spacer 70 as seen in FIG. 8 may be utilized (step 122a).

To fabricate an intraoral orthosis device 2 with releasable fastener 2l as shown in FIG. 2, a technician attaches the releasable fastener 21 to the crown of mandibular bite tray 4 and another releasable fastener 21 to the crown of the maxillary bite tray 5 (step 120b)

A variety of different permutations of the invention is contemplated, and not meant to be limited by this disclosure. The present invention is not limited to the preferred embodiments described in this section. The embodiments are merely exemplary, and one skilled in the art will recognize that many others are possible in accordance with this invention. Having now generally described the invention, the same will be more readily understood through references to the above descriptions and drawings, which are provided by way of illustration, and are not intended to be limiting of the present invention, unless so specified. Any element in a claim that does not explicitly state “means” for performing a specified function or “step” for performing a specified function, should not be interpreted as a “means” or “step” clause as specified in 35 U.S.C. §112.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings can be replaced by alternative features serving the same, equivalent, or similar purpose, unless expressly stated otherwise. Thus, unless expressly stated otherwise, each feature disclosed is one example only of a generic series of equivalent or similar features.

What is claimed:

1. An intraoral orthosis device comprising:
(a) a maxillary bite tray and a mandibular bite tray;
(b) a telescoping arm connecting the maxillary bite tray and the mandibular bite tray;
(c) the telescoping arm comprising an arm screw, an arm sleeve, and an arm rod; the arm sleeve having an interiorly threaded cylindrical portion and an interiorly smooth cylindrical portion;
wherein, the arm screw attaches to the interiorly threaded portion;
wherein, the arm rod attaches to the interiorly smooth cylindrical portion such that the arm rod rotates and moves freely.

2. The device as recited in claim 1, wherein at least one spacer is located between said telescoping arm and said maxillary bite tray and said mandibular bite tray, wherein the spacer creates a clearance between said telescoping arm and said maxillary bite tray and said mandibular bite tray; wherein the spacer has a bottom, and a cylindrical pivot; wherein said telescoping arm attaches to the cylindrical pivot and said telescoping arm is free to rotate and pivot.

3. The device as recited in claim 1, wherein said arm sleeve has an inscription; wherein the inscription provides information to a user on adjusting the telescoping arm.

4. The device as recited in claim 1, wherein said telescoping arm is made of stainless steel.

5. The device as recited in claim 1, wherein said arm sleeve has an adjustment port.

6. The device as recited in claim 1, wherein said arm rod length varies between 0.5 mm to 40 mm.

7. An intraoral orthosis device comprising:
(a) a maxillary bite tray and a mandibular bite tray;
(b) an anchor having a base and a shaft, the anchor is at least partially embedded in the maxillary bit tray and the mandibular bite tray;
wherein, the base consists of at least one petal extending from the longitude axis of the anchor.

8. The device as recited in claim 7, wherein said maxillary bite tray and said mandibular bite tray are connected by a telescoping arm;
wherein a spacer is located between the telescoping arm and said anchor; wherein the spacer creates a clearance between said arm and said maxillary bite tray and said mandibular bite tray; wherein the spacer has a bottom, and a cylindrical pivot; wherein said arm attaches to the cylindrical pivot and said arm is free to rotate and pivot.

9. The device as recited in claim 8, wherein said bottom distance varies between 0.5 mm to 20 mm.

10. The device as recited in claim 8, wherein a transition is located between said bottom and said cylindrical pivot; wherein the transition is fillet.

11. The device as recited in claim 8, wherein said anchor and said spacer is manufacture from stainless steel.

12. A method for manufacturing an intraoral orthosis device comprising the following steps:
(a) forming a cast of a user's teeth;
(b) placing a thermoplastic film over the cast;
(c) applying a heat and a vacuum to thermoplastic film; whereby the thermoplastic film conforms to the cast creating a bite tray;
(d) removing the bite tray from the cast and attaching the cast to a articulator; the articulator is attached to a sander;
(e) placing the crown of the cast against an analyzer creating a first angle and locking the articulator; whereby the first angle has a vertex similar to a temporomandibular joint;
(f) the bite tray is repositioned on the cast, and the bite tray is sanded;
(g) the process is repeated such that the method creates both a maxillary bite tray and a mandibular bite tray.

13. The method of claim 12 further comprising; attaching an anchor to said cast before placing said thermoplastic film over said cast; wherein the anchor is embedded into said thermoplastic film when said heat and said vacuum is applied to said thermoplastic film.

14. The method of claim 12 further comprising; attaching a telescoping arm to connect said maxillary bite tray and said mandibular bite tray.

15. The method of claim 12 further comprising; building a platform on said cast before placing said thermoplastic film over said cast; wherein said platform fuses together with said thermoplastic film when said heat and said vacuum is applied to said thermoplastic film.

16. The method of claim 12 further comprising; forming said thermoplastic material with a forming tool while said thermoplastic material is malable.

17. The method of claim 12 further comprising; said analyzer sets said first angle between 0.5 to 10 degrees.

18. The method of claim 12 further comprising; attaching a releasable fastener to the crown of said maxillary bite tray and said mandibular bite tray.

19. A method for manufacturing an intraoral orthosis device comprising the following steps:
(a) utilizing a telescoping arm to connect a maxillary bite tray and a mandibular bite tray; whereby the telescoping arm is attached to the maxillary bite tray and the mandibular bite tray by an anchor and an attachment screw; the telescoping arm comprising an arm screw, an arm sleeve, and an arm rod; the length of the arm rod varies; the arm sleeve having an interiorly threaded cylindrical portion and an interiorly smooth cylindrical portion; wherein, the arm screw attaches to the interiorly threaded portion; wherein, the arm rod attaches to the interiorly smooth cylindrical portion such that the arm rod rotates and slide freely;
wherein, to obtain the best fit by the user, the user adjust the length of the telescoping arm by twisting the arm screw into or out of the arm sleeve or by utilizing an extended arm road or a reduced arm rod;
(b) a spacer is located between the telescoping arm and the anchor; wherein the spacer has a bottom, and a cylindrical pivot; wherein said telescoping arm attaches to the cylindrical pivot and the telescoping arm is free to rotate and pivot; wherein the spacer creates a clearance between said arm and said maxillary bite tray and said mandibular bite tray;
wherein, to obtain the best fit by the user, the user adjust the clearance by utilizing an elongated spacer or a petite spacer.
